(19) United States
(12) Patent Application Publication
(10) Pub. No.: US 2021/0128041 A1
(43) Pub. Date: May 6, 2021

(54) Ergometer

(71) Applicants: UNIVERSITE DE BORDEAUX, Bordeaux (FR); CENTRE NATIONAL DE LA RECHERCHE SCIENTIFIQUE - CNRS, Paris (FR); INSTITUT POLYTECHNIQUE DE BORDEAUX, Talence (FR); ECOLE NATIONALE SUPERIEURE DES ARTS ET METIERS, Paris (FR)

(72) Inventors: Jocelyn SABATIER, Villenave d'Ornon (FR); Frédéric BOS, Libourne (FR); Adrien CAPORALE, Lastrene (FR); Ludovic DELHOMME, Arveyres (FR); Anne THÉVENOUX, Bordeaux (FR); Fanny MUNSCH, Bordeaux (FR)

(73) Assignees: UNIVERSITE DE BORDEAUX, Bordeaux (FR); CENTRE NATIONAL DE LA RECHERCHE SCIENTIFIQUE - CNRS, Paris (FR); INSTITUT POLYTECHNIQUE DE BORDEAUX, Talence (FR); ECOLE NATIONALE SUPERIEURE DES ARTS ET METIERS, Paris (FR)

(21) Appl. No.: 16/472,029

(22) PCT Filed: Dec. 20, 2017
(86) PCT No.: PCT/FR2017/053754

§ 371 Date: Oct. 25, 2019

(30) Foreign Application Priority Data
Dec. 20, 2016 (EP) 16306747.3

(51) Int. Cl. A61B 5/22; A61B 5/055; A63B 21/008; A63B 21/00; A63B 22/06; A63B 24/00

(52) U.S. Cl. CPC A61B 5/222; A61B 5/055; A63B 21/0083; A63B 21/00069; A63B 2220/51; A63B 24/0062; A63B 21/4034; A63B 21/4035; A63B 2022/0652; A63B 22/0605

(57) ABSTRACT

The present invention concerns an ergometer (10) comprising at least one training assembly, each assembly comprising: a training element (11), -at least one hydraulic cylinder (12) comprising a pressure chamber (32), said training element (11) being linked to said at least one hydraulic cylinder (12) such that a hydraulic pressure value applied in the pressure chamber (32) of said or at least one of said corresponding cylinders determines a force opposing the movement of said training element (11) by a user, and at least one control unit (13, 14) for supplying the pressure chamber (32) of said or one corresponding hydraulic cylinder (12) with pressurised hydraulic fluid such that said pressure chamber (32) has a chosen pressure value during at least a part of the muscle exercises carried out by the user, said control unit (13, 14) being linked to said pressure chamber (32) of the corresponding hydraulic cylinder (12) by a hydraulic fluid supply circuit.

### BACKGROUND OF THE INVENTION

### Field of the Invention

The present invention relates to the field of ergometers.

In particular, it relates to an ergometer enabling exercises to be performed by an individual placed in a prone position in a magnetic resonance imaging type (MRI) device in order to assess the mechanical properties of one or more groups of muscles of this individual.

In a non-exhaustive manner, this invention is also applicable in various other medical fields, such as cardiology, neurology, angiology or even orthopedics.

More generally, the present invention can have numerous relevant applications in the medical field, in biomedical research and in basic research.

### Technological Background

Tests can be conducted on an individual in order to assess their muscular, neurological, cardiac, venous and arterial, orthopedic functions, etc.

From a biomedical perspective, these studies can be useful for investigating the functioning that governs the locomotion of healthy or sick subjects. For example, in a patient who has suffered an accident affecting the central or peripheral motor functions, an ergometer could allow the loss or the deterioration of these functions to be assessed. Similarly, the use of an ergometer could allow the handling of the rehabilitation of the patient to be adjusted.

According to another example, an ergometer allows a bedridden person or an elderly person to be studied with respect to the muscular loss that is likely to restrict their mobility. The idea is to better understand the factors that cause this loss in order to anticipate it and to implement suitable handling with a view to minimizing this loss, for example, with the help of a physiotherapist.

Currently, the ergometers of the prior art offer limited uses compared to the requirements.

Thus, due to their constituent materials, some ergometers cannot be used on imaging equipment, which are nevertheless essential in diagnosing motor disorders.

Indeed, it is known that magnetic resonance imaging (MRI) allows the motor functions to be viewed on a muscular and articular level and, in functional MRI, in a cerebral level.

However, since an MRI appliance is a machine emitting magnetic fields, the applicants have noted that associating an ergometer and a magnetic resonance imaging appliance is extremely difficult.

It has been noted that, even if an ergometer of the prior art comprises parts made of non-ferromagnetic material, the images that are acquired are not free of artefacts. In particular, it has been seen that at least the electrical system of the ergometer is capable of disrupting the operation of the MRI appliance.

However, one imperative is that the ergometer has no influence whatsoever on the quality of the images acquired by the medical imaging appliance.

Therefore, the ergometer must be constructed using non-ferromagnetic parts that are verified as not being able to generate disruptions in the magnetic field, which would lead to malfunctions both in the ergometer and in the MRI appliance.

Most ergometers that are available on the market overcome these disruptions by moving the activity on the ergometer to another room.

However, this solution does not allow the moving human body to be studied by MRI.

The applicants have also seen that the existing ergometers are often bulky, and are difficult to move and handle by a single qualified operator.

Thus, the ergometer can be placed at the end of the imaging device, by being fixedly connected to the table so as not to move when an individual performs exercises.

However, such an arrangement of the ergometer is incompatible with the need to quickly handle a patient placed in the imaging device and in need of care.

Furthermore, some ergometers can only be used for one member at a time or, if they allow exercises to be performed with the two members, they only aim for the same resistance to be applied to each member.

This is the case, for example, for an ergometer comprising a pedal device, in which the force is transferred to the patient by a brake disk without control of the braking torque.

Other ergometers are difficult to use in the prone position.

Finally, none of them allows image capture to be synchronized with the execution of the required physical exercise.

Furthermore, these ergometers of the prior art exhibit static and kinetic friction that lead to energy losses, which are therefore likely to falsify the measurements.

Therefore, there is an urgent requirement for an ergometer that is capable of examining a patient or an individual in a magnetic resonance imaging appliance, the original operating principle of which overcomes the various disadvantages mentioned above.

### Subject Matter of the Invention

The aim of the present invention is an ergometer with a simple design and operating mode that is ergonomic, multi-purpose, configurable and that can be synchronized with the control station in order to perform, using imaging, diagnostics, therapeutic follow-up, biomedical research and basic research projects.

A further aim of the present invention is that such an ergometer allows precise measurement of the forces, the positions and the powers exerted by one or more groups of muscles, such as the muscles for bending the foot, of an individual.

Another aim of the present invention is that such an ergometer is capable of operating in a magnetic resonance imaging system (MRI), without creating artefacts in the acquired data.

A still further aim of the present invention is that such an ergometer is particularly easy to handle.

The present invention also relates to a measurement appliance for performing medical and/or sporting diagnostics comprising a magnetic resonance imaging system (MRI) and such an ergometer.

### BRIEF DESCRIPTION OF THE INVENTION

To this end, the invention relates to an ergometer comprising at least one training assembly, each assembly comprising:
a training element;
at least one hydraulic cylinder comprising a pressure chamber;
said training element being linked to said at least one hydraulic cylinder such that a hydraulic pressure value applied in the pressure chamber of said cylinder, or of at least one of said corresponding cylinders, determines a force opposing the movement of said training element by a user; and
at least one control unit for supplying the pressure chamber of said hydraulic cylinder, or of a corresponding hydraulic cylinder, with pressurized hydraulic fluid such that said pressure chamber has a selected pressure value during at least part of the muscle exercises performed by the user, said control unit being linked to said pressure chamber of the corresponding hydraulic cylinder by a hydraulic fluid supply circuit.

“Hydraulic fluid” is understood to be a non-viscous fluid defined by a kinematic viscosity coefficient below 1.3·10⁻¹⁰ m²/s and a fluid compressibility coefficient above 2·10⁹ N/m².

Solely by way of an illustration, the hydraulic fluid is an aqueous product, i.e. a water-based liquid. Advantageously, mineral or synthetic oils will not be implemented.

The use of air, for its part, results in regulation control problems due to the simultaneous presence of dry friction, compressibility phenomena and high air circulation speeds.

For each training assembly, with the piston rod of the hydraulic cylinder being linked to the corresponding training element, the pressure of the hydraulic fluid present in the pressure chamber determines the resistant force that must be overcome by the user in order to move this training element. With the control unit supplying this chamber with pressurized hydraulic fluid via a hydraulic fluid supply circuit, the force to be produced is applied to the user only by a hydraulic distribution.

Extremely good control of the resistance applied to the individual when performing exercises is thus obtained, whilst having the possibility of easily returning to a standard comfort position.

Since the drive element is a pedal, this can have a shoe for accommodating the foot of the user, said shoe being fixed on the pedal in order to securely retain the foot with the pedal.

Alternatively, or additionally, a set of straps also can be provided to retain the foot on the pedal.

Advantageously, the ergometer according to the present invention comprises means for adjusting each training element relative to a support supporting the one or more training elements. These adjustment means can, for example, allow the height and/or the depth of the position of each training element to be adjusted in order to adjust the ergometer according to the size of the user, and in particular the size of the leg of the user, and/or allow the incline of the foot on the support surface of the plate to be adjusted.

Various particular embodiments of this ergometer can be contemplated, with each having its particular advantages and being subject to numerous possible technical combinations.

Each training assembly comprises one training element, two hydraulic cylinders and, for each hydraulic cylinder, one corresponding control unit for supplying the pressure chamber of said hydraulic cylinder with pressurized hydraulic fluid.

Preferably, a first one of these hydraulic cylinders linked to said training element is supported by a translationally movable plate, said plate being rigidly connected to the piston rod of the second hydraulic cylinder.

This ergometer comprises two training assemblies, each training assembly being configured to measure at least the force applied by each of the members of the user.

By way of an illustration, this user can be a voluntary patient.

Of course, the ergometer can comprise a means for locking the training element of one of the two assemblies when seeking to perform tests on a single member of the user.

By way of an illustration, with this ergometer comprising two training assemblies, each training assembly comprises a set of sensors intended to measure the force applied by each of the members of the user and the power of the training (pedaling power).

With this ergometer comprising a plurality of training elements, said control units are separate such that said ergometer permits the application of unsymmetrical forces to the user.

Such an embodiment allows modulation of the forces on each member of a user. In particular, such an embodiment allows modulation of the forces until the limit from which the corresponding member can no longer perform the exercise is determined. Preferably, with the ergometer comprising two training assemblies, the pressure value selected in the pressure chamber of the hydraulic cylinder of each assembly is different. A non-symmetry is thus applied to the training elements.

Each training element is a pedal or a handle.

Each sub-assembly, or group, comprising a training element and said corresponding hydraulic cylinder, is mounted on a platform, which is mounted on a movable carriage.

Preferably, this platform is itself movable between a deployed position and at least one retracted position.

This carriage allows the handling and the movement of the one or more sub-assemblies to be simplified. It is thus possible to position the ergometer at the two ends of a bed type table of an MRI system or of a scanner or of a PET scan.

Preferably, this platform comprises a suction position retention device for locking said platform in position when the ergometer is operating.

Solely by way of an illustration, this position retention device can comprise a plurality of suction cups linked to a suction or vacuum system.

Each sub-assembly, or group, comprising a training element and said corresponding hydraulic cylinder, also comprises a force sensor for measuring the forces and/or a pressure sensor for determining the pressure of the fluid in said pressure chamber.

In order to measure the force exerted when the foot of the user is bent, the force sensor advantageously is designed to be of the piezoresistive or gauge type.

Advantageously, the ergometer according to the present invention comprises a computer processing unit that is capable of receiving and processing the measurement signals originating from the one or more sensors in order to determine the force exerted by the muscles used in the movements exerted by the user, with said user being able to be a patient.

Each hydraulic fluid supply circuit is at least partly flexible to allow adjustment of the position of each sub-assembly, or group, comprising a training element and said corresponding hydraulic cylinder relative to its control unit.

Each fluid supply circuit comprises a quick disconnect system allowing the sub-assembly, or group, comprising a training element and said corresponding hydraulic cylinder to be separated from its control unit.

Unlike the ergometers on the market, such a quick disconnect system allows emergency separation of the sub-assembly, or group, comprising a training element and said corresponding hydraulic cylinder from its control unit.

Quick intervention is thus possible when a patient needs to be withdrawn from the medical imaging appliance in the event of complications occurring during the examination.

Each control unit comprises a hydraulic cylinder comprising a pressure chamber, said pressure chamber being in fluid communication with said hydraulic fluid supply circuit, and the piston rod of said hydraulic cylinder being connected to a drive mechanism configured to exert a predetermined hydraulic pressure in the pressure chamber of the hydraulic cylinder of said control unit.

Preferably, this drive mechanism comprises a stepper motor having a driveshaft, the rod of said piston being rigidly connected to a rack, or serrated cam, linked by a transmission to the shaft of said stepper motor, which thus enables the movement of the rack and the pressurization of the fluid in said pressure chamber.

With the stepper motor having a driveshaft, said driveshaft is linked by a substantially play-free toothed wheel to said rack. Said rack is thus driven by the stepper motor in order to determine the pressure value of the hydraulic fluid in the hydraulic cylinder.

It also comprises a motor control unit designed to supply the stepper motor with operating signals, which unit preferably can be programmed according to at least one parameter such as the resistance, the movement and/or the power that must be provided by each training element when tests are performed by the user.

Preferably, this motor control unit can comprise at least one computer program for automatically varying the force opposing the movement of the training element by the user, for example, according to a curve of predefined force values.

This control unit allows application, as a function of the angular position of the driveshaft, of a movement of the rack and, consequently, a hydraulic fluid pressure value through the linear movement of the piston rod of the corresponding hydraulic cylinder.

Advantageously, this drive mechanism comprises a motor current measurement device for determining the torque exerted by the shaft thereof in order to retain said rack in position.

A non-magnetic screen externally surrounds at least one part of each control unit, with the rest of the ergometer being made of a non-magnetic material.

Such a screen forms a shield advantageously allowing the falsification of the measurements performed by an imaging device such as an MRI system to be prevented.

At least some parts can be produced by 3D printing, for example, using plastic materials such as thermoplastics. By way of an example, the material can be selected from acrylonitrile butadiene styrene (ABS), polycarbonate (PC), polyamides, polypropylene (PP). Thus, some of the parts of the hydraulic cylinders, such as the piston, advantageously can be produced by 3D printing in polycarbonate (PC). Methods for machining and/or cutting and/or molding also can be used to manufacture these parts.

In order to strengthen wear parts, inserts can be used that are made, for example, of aluminum, or are aluminum-based, of brass, of copper, or are copper-based.

Similarly, each supply circuit advantageously will be produced by implementing a flexible pipe made of plastic or rubber material.

The present invention also relates to a measurement appliance for performing diagnostics, such as medical and/or sporting diagnostics, and for research.

According to the invention, this appliance comprises a magnetic resonance imaging system (MRI) and an ergometer as previously described.

Such a measurement appliance thus allows an individual to perform exercises by applying a force to them during a medical imaging session of the MRI type.

The present invention also relates to a measurement method, in which, with each training assembly comprising one training element, two hydraulic cylinders and, for each hydraulic cylinder, one corresponding control unit for supplying the pressure chamber of this hydraulic cylinder with pressurized hydraulic fluid, a selected pressure value is applied, for at least some of the muscle exercises performed by the user, in the pressure chamber of only one of said cylinders, with the other cylinder being left free in order to stress different parts of the body of the user.

Preferably, a first one of these hydraulic cylinders linked to said training element is supported by a translationally movable plate, said plate being rigidly connected to the rod of the piston of the second hydraulic cylinder.

The present invention also relates to a measurement method, according to which a plurality of items of information is obtained in real time that characterizes the physiological or physiopathological functioning of a user performing exercises on an ergometer as described above. This user can, of course, be a patient or a volunteer.

Solely by way of an illustration, this information can originate from a plurality of images obtained by magnetic resonance imaging (MRI).

Advantageously, this method can be used to study the activated motor zones of the brain or the locomotor device of an individual, when performing exercises, as a function of the applied resistance.

It is also applicable in the fields of cardiology, neurology, angiography or even orthopedics.

Thus, the aim of the present invention, due to its various functional and structural aspects described above, allows precise and reliable measurement of the force produced by one or more groups of muscles, whilst avoiding any disruption in the measurements that are performed.

### BRIEF DESCRIPTION OF THE DRAWINGS

Further advantages, aims and particular features of the present invention will become apparent from the following description, which is provided by way of a non-limiting explanation, with reference to the accompanying drawings, in which:

FIG. 1 schematically shows an ergometer for performing force tests on a patient according to a first embodiment of the present invention;

FIG. 2 is an exploded view of a control unit of the ergometer of FIG. 1;

FIG. 3 is a longitudinal section view of the control unit of FIG. 2 and of the cylinder, with the pressure chamber of the control unit being linked to the pressure chamber of the cylinder via a hydraulic fluid supply circuit;

FIG. 4 is a schematic view of the ergometer showing a section outside the magnetic field and a section inside the magnetic field;

FIG. 5 schematically shows the ergometer of FIG. 1 disposed at the front of an MRI system (FIG. 5a) and at the rear of an MRI system (FIG. 5b);

FIG. 6 shows a partial view of an ergometer for performing force tests on a patient according to a second embodiment of the present invention.

### DETAILED DESCRIPTION OF EMBODIMENTS OF THE INVENTION

Firstly, it is to be noted that the figures are not to scale.

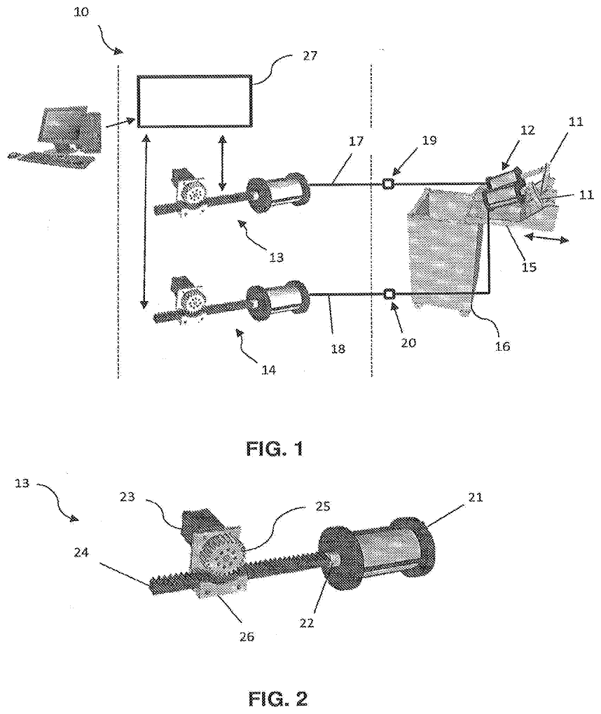

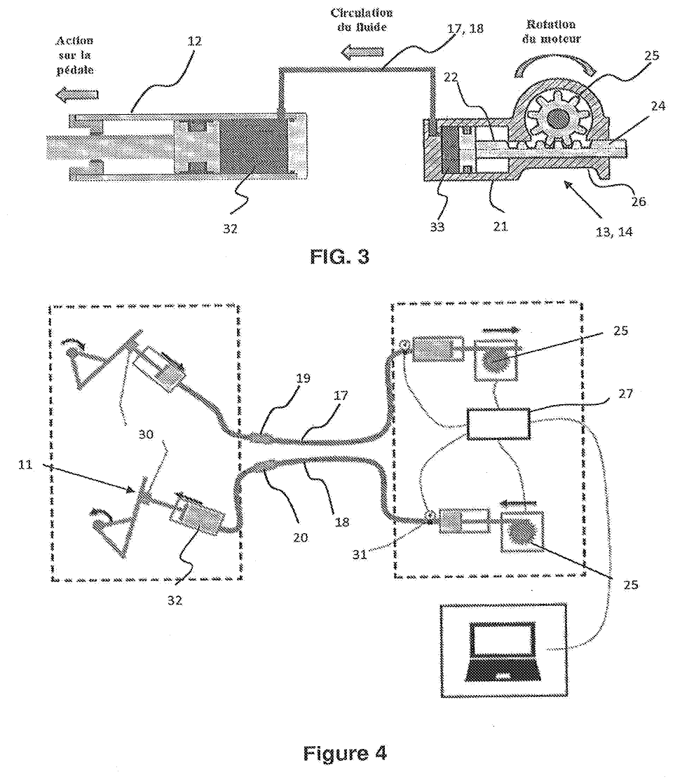

An ergometer according to one embodiment of the present invention will now be described hereafter with joint reference to FIGS. 1 to 5.

In the embodiment described herein, the ergometer 10 comprises two training assemblies that are separate and are placed substantially parallel to each other.

Each training assembly comprises a pedal 11 linked to a hydraulic cylinder 12 comprising a pressure chamber 32 such that a hydraulic pressure value applied in this pressure chamber 32 determines a force opposing the movement of the corresponding pedal 11 by the foot of a user.

The ergometer also comprises a force sensor 30 placed on each pedal 11 to measure the applied forces. This information can be used to control the force or the power supplied by the pedal 11.

The ergometer can also comprise an angular movement sensor (not shown) placed on each pedal 11 to determine the speed of the pedals and therefore the pedaling power. This information can be used to control the power, but also the position, of the pedals.

Each assembly also comprises a control unit 13, 14 for supplying this pressure chamber 32 of the corresponding hydraulic cylinder 12 with pressurized hydraulic fluid such that said pressure chamber has a selected pressure value for at least some of the exercises performed by this user.

Each sub-assembly comprising a pedal 11 and its corresponding hydraulic cylinder 12 and force sensor 30 is mounted on the same platform 15.

This platform 15 is itself supported by a carriage 16 that is movable to enable the movement thereof. This carriage 16, which is typically placed at the end of the table of the magnetic resonance imaging system (MRI), thus can be easily handled by an operator.

The platform 15 is movably mounted on the carriage 16, via slides 34, 35, between a deployed position, in which the platform 15 is placed on the MRI table in order to position the pedals on this table, and a retracted position, in which the platform 15 is removed from the end of this table.

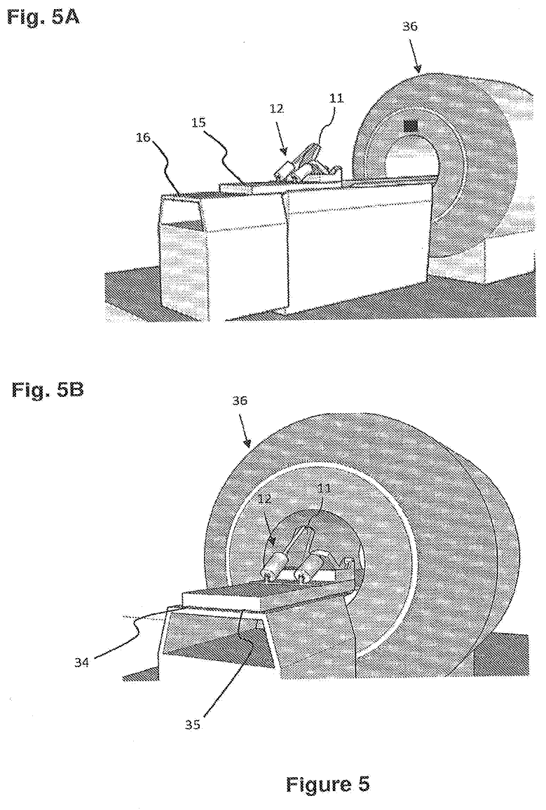

As shown in FIGS. 5A and 5B, the carriage supporting the pedal can be placed at the front of the MRI 36 (FIG. 5A) or at the rear of the MRI 36 (FIG. 5B).

This platform 15 also comprises a plurality of suction cups linked to a suction device (not shown) to enable easy positional locking of this platform on the MRI table 36 when the ergometer 10 is used.

Each control unit 13, 14 is linked to the pressure chamber 32 of the corresponding hydraulic cylinder 12 by a flexible hydraulic fluid supply circuit 17, 18.

These supply circuits 17, 18 thus allow adjustment of the position of the carriage 16 relative to the control units 13, 14 that are placed at a distance from the tunnel of the magnetic resonance imaging system. Solely by way of an illustration, these control units 13, 14 can be placed several meters from the entrance of the mini-tunnel.

In the case of the MRI, the generated magnetic field decreases as a function of the distance, thus advantageously ensuring the absence of disruption generated by this magnetic field on the control units 13, 14.

Each hydraulic fluid supply circuit 17, 18 comprises a quick disconnect system 19, 20 for separating each sub-assembly from its corresponding control unit 13, 14. By way of an example, such a quick disconnect system 19, 20 comprises a sealed quick disconnect connector.

Each control unit 13, 14 comprises a hydraulic cylinder 21 comprising a pressure chamber 33, with this pressure chamber being in fluid communication with its hydraulic fluid supply circuit 17, 18.

The rod 22 of the piston of each hydraulic cylinder 21 is linked to a drive mechanism configured to exert a predetermined hydraulic pressure in the pressure chamber of the hydraulic cylinder 21 of said corresponding control unit 13, 14.

This drive mechanism in this case comprises a stepper motor 23 having a driveshaft, the rod 22 of the piston of the corresponding hydraulic cylinder being rigidly connected to a serrated cam 24.

This serrated cam 24 itself is linked by a toothed wheel 25 to the shaft of the stepper motor 23, which thus ensures the movement for the serrated cam 24 and the pressurization of the fluid in the pressure chamber 33 of the corresponding hydraulic cylinder.

A guide 26 allows linear movement of the serrated cam 24 to be provided.

An electronic unit 27 allows each stepper motor 23 to be controlled, with a programming unit (not shown) allowing, for example, the resistance applied to each pedal 11 to be varied by applying a determined hydraulic pressure in the hydraulic supply circuit 17, 18 extending between the pressure chambers of the two hydraulic cylinders 12, 21 of each drive system.

In order to avoid any disruption in the measurements performed by a magnetic resonance imaging system, a non-magnetic screen (not shown) externally surrounds each control unit 13, 14, with the rest of the ergometer being made of non-magnetic material.

The ergometer comprises a set of sensors 30, 31 disposed on the pedals, the cylinders and the motors.

According to one embodiment, a pair of fluid pressure sensors is disposed in the chambers, a pair of position and speed sensors is disposed on the pedals and a pair of current sensors is disposed on the motors.

According to one operating mode, the information from the sensors 30, 31 is transmitted to the electronic unit 27, which retransmits it to a control station provided with a display screen.

According to the information displayed on the control station, an operator can send a setpoint to control the force returned by the motors to the pedals via the control units 13, 14, in order to adjust the position or the speed or the power at the pedal.

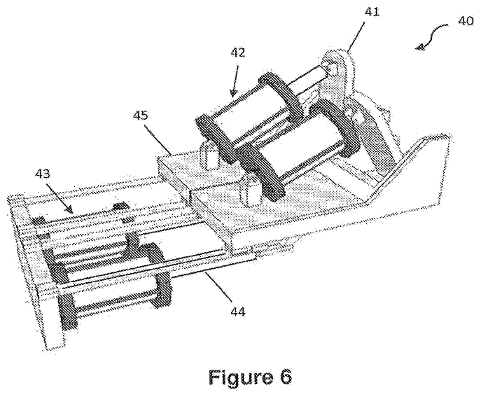

FIG. 6 shows a partial view of an ergometer 40 for performing force tests on a patient according to a second embodiment of the present invention.

This ergometer 40 comprises two independent training assemblies, which are distinct and are placed substantially parallel to each other. Each training assembly comprises a pedal 41, as well as two hydraulic cylinders 42, 43, with each cylinder conventionally comprising a pressure chamber and a piston rod 44.

The pressure chamber of each cylinder 42, 43 of each training assembly is linked to its own control unit (not shown) such that the pressure chamber of each cylinder 42, 43 is autonomously supplied with hydraulic fluid.

With such an embodiment, it is advantageously possible to exercise a distinct part of each leg of the patient by applying a selected pressure value in the pressure chamber of one of the two cylinders 42, 43 and by leaving the other cylinder free. Several combinations are thus possible with the two training assemblies.

More specifically, the pedal 41, or support for the feet of the patient, of each training assembly is mounted with a first cylinder 42, to which it is linked, on a translationally movable plate 45, in this case a plate with slides.

This plate 45, which is rigidly connected to the piston rod of each second cylinder 43 of the drive assemblies, is moved by these second cylinders 43.

When the user wants to stress the thigh muscles, for example, a resistant force is applied by the second cylinders 43, with a selected pressure value then being applied by the corresponding control unit in the pressure chamber of each of these second cylinders 43. The first cylinders 42 directly linked to the pedals 41 and associated with the heels of the patient are then left free. It is the entire plate 45 that moves.

When the user wishes to stress their heels, the piston rods 44 of the second cylinders 43, rigidly connected to the plate 45, are returned such that the plate 45 then covers these second cylinders 43. A determined pressure value, for at least some of the exercises performed by this user, is applied in the pressure chamber of each inclined pressure cylinder 42, which then push on the pedals 41 activated by this user.

1. An ergometer comprising at least one training assembly, each training assembly comprising:
a training element;
at least one hydraulic cylinder comprising a pressure chamber;
said training element being linked to said at least one hydraulic cylinder such that a hydraulic pressure value applied in the pressure chamber of said corresponding cylinder, or of at least one of said corresponding cylinders, determines a force opposing the movement of said training element by a user; and
at least one control unit for supplying the pressure chamber of said hydraulic cylinder, or of a corresponding hydraulic cylinder, with pressurized hydraulic fluid such that said pressure chamber has a selected pressure value during at least part of the muscle exercises performed by the user, said control unit being linked to said pressure chamber of the corresponding hydraulic cylinder by a hydraulic fluid supply circuit.

2. The ergometer as claimed in claim 1, wherein each training assembly comprises one training element, two hydraulic cylinders and, for each hydraulic cylinder, one corresponding control unit for supplying the pressure chamber of said hydraulic cylinder with pressurized hydraulic fluid.

3. The ergometer as claimed in claim 2, wherein a first hydraulic cylinder linked to said training element is supported by a translationally movable plate, said plate being rigidly connected to a piston rod of the second hydraulic cylinder.

4. The ergometer as claimed in claim 1, wherein it comprises two training assemblies, each training assembly comprising a set of sensors intended to measure the force applied by each of the members of the user and the power of the training.

5. The ergometer as claimed in claim 1, wherein said ergometer comprises a plurality of training assemblies, said control units are distinct such that said ergometer permits the application of unsymmetrical forces to the user.

6. The ergometer as claimed in claim 1, wherein each training element is a pedal or a handle.

7. The ergometer as claimed in claim 1, wherein each sub-assembly comprising a training element and said corresponding hydraulic cylinder is mounted on a platform, which preferably can move between a deployed position and at least one retracted position, which platform is mounted on a movable carriage.

8. The ergometer as claimed in claim 7, wherein said platform comprises a suction position retention device for locking said platform in position when the ergometer is used.

9. The ergometer as claimed in claim 1, wherein each sub-assembly comprising a training element and said corresponding hydraulic cylinder also comprises a force sensor for measuring the forces and/or a pressure sensor for determining the pressure of the fluid in said pressure chamber.

10. The ergometer as claimed in claim 1, wherein each hydraulic fluid supply circuit is at least partly flexible to enable an adjustment of the position of each sub-assembly comprising a training element and said corresponding hydraulic cylinder relative to its control unit.

11. The ergometer as claimed in claim 1, wherein each fluid supply circuit comprises a quick disconnect system allowing the sub-assembly comprising a training element and said corresponding hydraulic cylinder to be separated from its control unit.

12. The ergometer as claimed in claim 1, wherein each control unit comprises a hydraulic cylinder comprising a pressure chamber, said pressure chamber being in fluid communication with said hydraulic fluid supply circuit and a piston rod of said hydraulic cylinder being connected to a drive mechanism configured to exert a predetermined hydraulic pressure in the pressure chamber of the hydraulic cylinder of said corresponding control unit.

13. The ergometer as claimed in claim 12, said drive mechanism comprises a stepper motor having a driveshaft, the piston rod being rigidly connected to a rack linked by a transmission to the shaft of said stepper motor, which thus enables the movement of the rack and the pressurization of the fluid in said pressure chamber.

14. The ergometer as claimed in claim 13, wherein it comprises a motor current measurement device for determining the torque exerted by the shaft thereof in order to retain said rack in position.

15. The ergometer as claimed in claim 1, wherein a non-magnetic screen externally surrounds at least one part of each control unit, the rest of the ergometer being made of a non-magnetic material.

16. A measurement appliance for performing diagnostics and for research, wherein it comprises a magnetic resonance imaging system (MRI) and an ergometer as claimed in claim 1.

17. A measurement method, wherein, using an ergometer as claimed in claim 2, a selected pressure value is applied, for at least part of the muscular exercises performed by the user, in the pressure chamber of only one of said cylinders, the other cylinder being left free in order to stress different parts of the body of the user.

18. A measurement method wherein a plurality of items of information is obtained in real time that characterizes the physiological or physiopathological functioning of a user performing exercises on an ergometer as claimed in claim 1.
